(19) United States
(12) Patent Application Publication
(10) Pub. No.: US 2005/0215935 A1
(43) Pub. Date: Sep. 29, 2005

(54) Method and apparatus for improving the circulation of blood in the extremities of a person with diabetes

(75) Inventor: Marcus Ritter, Boise, ID (US)

Correspondence Address: STEPHEN M. NIPPER;DYKAS, SHAVER & NIPPER, LLP, PO BOX 877, BOISE, ID, 83701, US

(73) Assignee: My Soxx Development Co., LLC, Boise, ID (US)

(21) Appl. No.: 11/026,605

(22) Filed: Dec. 30, 2004

Related U.S. Application Data
(60) Provisional application No. 60/555,445, filed on Mar. 23, 2004.

(51) Int. Cl. A61F 13/00

(52) U.S. Cl. 602/65

(57) ABSTRACT

A foot and leg warmer or foot covering configured for wearing by a user, the foot and leg warmer having a sole portion, front portion, rear portion, upper front portion, and an adjustable attachment configured for allowing the user to fit the foot and leg warmer to the user's body. The foot covering configured for attaching above a wearer's calf muscle, thereby using the warmth of the wearer's calf muscle to heat air trapped within the foot covering and thereby heats the wearer's lower extremities.

### PRIORITY

This application claims the priority date of the provisional application entitled Foot and Leg Warmers filed by Marcus Ritter on Mar. 23, 2004 with application Ser. No. 60/555,445.

### BACKGROUND OF THE INVENTION

1. Field of the Invention

The present invention generally relates to clothing, and more particularly relates to footwear especially configured to be worn by individuals suffering from circulation problems.

2. Background Information

It is estimated that as many as twenty million people in the United States suffer from diabetes. One of the complications often associated with diabetes is poor circulation within the extremities. This poor circulation often leads to other conditions and problems such as nervous system damage. It is estimated that sixty to seventy percent of people with diabetes have mild to severe forms of nervous system damage. Such nervous system damage often includes impaired sensation or pain within one's feet, such as constrictions or other pressure contact upon one's lower extremities (lower legs, feet, and toes). This can often result in severe pain and discomfort to the diabetic.

There are a number of different types of diabetic socks on the market; socks that are specially created for diabetics and those with sensitive feet. Additionally, a number of other foot coverings (some with medical purposes and others without) are shown in the prior art.

One such example is the “Rooke Boot” shown in U.S. Pat. No. D326,556 (Rooke et al.). As Applicant understands it from his reading of documents on the Internet, a Rooke Boot® is an insulated boot that “fits snugly” on the foot of a wearer. The Rooke Boot® uses a number of hook-and-loop (i.e., Velcro®) fasteners, which allow the boot to be opened up for simplifying the putting on/taking off of the boot. These fasteners allowing the boot to be snugly attached. The Rooke Boot® does not show a foot covering configured for loosely fitting a wearer's foot, thereby defining an air space generally surrounding said foot, nor does the Rooke Boot® extend above the person's calf muscle, nor is the Rooke Boot® configured for constricting above the person's calf muscle thereby trapping warm air within said air space.

The Sloan patent (U.S. Pat. No. 6,665,883) shows an over sock comprised of an elastic material stretchable over a conventional sock. As such, the Sloan device is not loose fitting.

The prior art does show a number of loose fitting foot coverings, typically for the purpose of protecting one's shoes from hazardous materials (blood, chemicals, etc.) or other purposes. For instance, the patents to Brandon (U.S. Pat. No. 4,918,839), Terry (U.S. Pat. No. 4,023,281), Maturaporn (U.S. Pat. No. 5,946,822), Price (U.S. Pat. No. 3,359,658), Ahern (U.S. Pat. No. 6,430,771), and Lennon et al. (U.S. Pat. No. 5,083,557). Such coverings configured for protecting and not for retaining heat, especially not using the heat of a calf muscle to heat trapped air.

What is needed is a diabetic sock that is loose fitting, comfortable, insulated, and creates a large pocket of warm air which is used to warm the wearer's feet and toes. Embodiments of the present invention solve these problems.

Additional objects, advantages and novel features of the invention will be set forth in part in the description which follows and in part will become apparent to those skilled in the art upon examination of the following or may be learned by practice of the invention. The objects and advantages of the invention may be realized and attained by means of the instrumentalities and combinations particularly pointed out in the appended claims.

### SUMMARY OF THE INVENTION

The present invention is a foot and leg warmer (foot covering) for use by individuals with circulatory disorders. This foot and leg warmer for wearing on a user's foot and/or legs. In the preferred embodiment, the foot and leg warmer comprises a sole portion for covering the sole of the wearer, a front portion for covering a wearer's shin, a rear portion for covering a wearer's calf area, an upper front portion for covering the top of a wearer's foot, and an adjustable attachment for enabling the warmer to be attached to a wearer's body. The adjustable attachment preferably a hook and loop style (Velcro®) strap for attaching the foot and leg warmer above the user's calf, thereby holding the foot and leg warmer up and for closing the foot and leg warmer thereby defining an air space therein. The warmth of the wearer's calf muscle is then used to warm the air trapped therein, this warm air then warming the user's lower extremities (feet and toes), thereby improving a wearer's circulation and comfort.

The purpose of the foregoing Abstract is to enable the United States Patent and Trademark Office and the public generally, and especially the scientists, engineers, and practitioners in the art who are not familiar with patent or legal terms or phraseology, to determine quickly from a cursory inspection, the nature and essence of the technical disclosure of the application. The Abstract is neither intended to define the invention of the application, which is measured by the claims, nor is it intended to be limiting as to the scope of the invention in any way.

Still other objects and advantages of the present invention will become readily apparent to those skilled in this art from the following detailed description wherein I have shown and described only the preferred embodiment of the invention, simply by way of illustration of the best mode contemplated by carrying out my invention. As will be realized, the invention is capable of modification in various obvious respects all without departing from the invention. Accordingly, the drawings and description of the preferred embodiment are to be regarded as illustrative in nature, and not as restrictive in nature.

### BRIEF DESCRIPTION OF THE DRAWINGS

FIG. 1 is a perspective view of a first embodiment of the present invention.

FIG. 2 is a perspective view of a second embodiment of the present invention.

FIG. 3 is a partial, cross-sectional side view of a third embodiment of the present invention.

FIG. 4 is a bottom view of a fourth embodiment of the present invention.

FIG. 5 is a chart showing a first clinical study of the benefits of the present invention.

FIG. 6 is a chart showing a second clinical study of the benefits of the present invention.

### DESCRIPTION OF THE PREFERRED EMBODIMENTS

While the invention is susceptible of various modifications and alternative constructions, certain illustrated embodiments thereof have been shown in the drawings and will be described below in detail. It should be understood, however, that there is no intention to limit the invention to the specific form disclosed, but, on the contrary, the invention is to cover all modifications, alternative constructions, and equivalents falling within the spirit and scope of the invention as defined in the claims.

While the present invention is particularly directed towards increasing the circulation of blood in the extremities of those suffering from diabetes, the present invention may likewise be useful in the treatment of neuropathy (nerve damage), arthritis, lupus, edema (swelling of the legs), sensitive feet, and poor leg circulation in general. The discussion of the present application herein is not thus intended to limit its use and/or application solely to the treatment of poor circulation in diabetics.

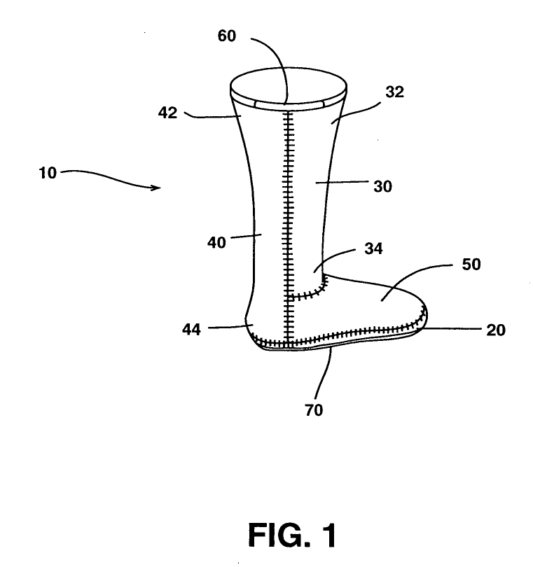

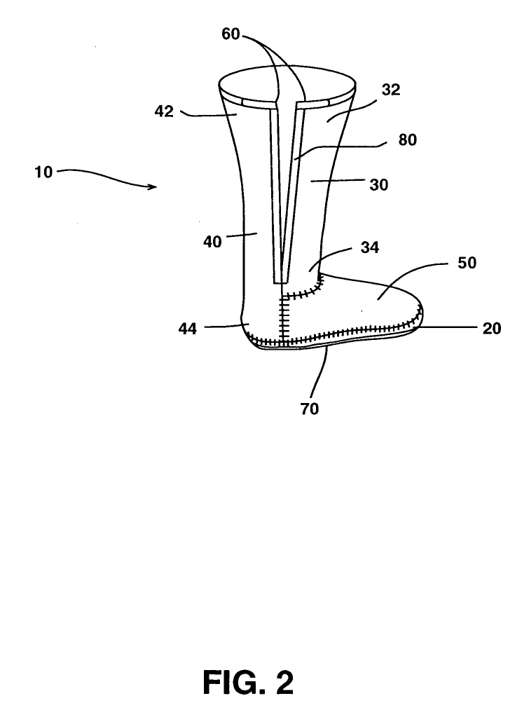

Referring initially to FIGS. 1-2, shown is a first embodiment of the present invention. These figures show the invented foot covering (foot and leg warmer) 10 comprising a sole portion 20, a front portion 30, a rear portion 40, an upper front portion 50, an adjustable attachment 60, and a nonskid sole 70. While this embodiment shows the foot covering 10 comprising a number of different components that are sewn together, it is expressly envisioned that more or less components could be present. For instance, the present invention could comprise a single piece molded into the appropriate shape, the front portion and the upper front portion could be a unitary piece, etc.

The foot covering 10 having a sole portion 20 that operates like the sole in typical footwear does, providing a first surface for contacting a user's foot and providing a second surface for contacting a ground surface. In order to provide the necessary level of comfort to a diabetic or other person suffering from poor circulation, the sole needs to be an integral unit and not have a bottom seam or other constriction that would irritate the skin of its wearer. The preferred embodiment of the present invention does exactly this, providing a sole without a bottom seam. The present invention is intended to be worn by a wearer the entire day and as such it is configured for having a sole.

In order to decrease the likelihood that a person wearing the present invention would slip on a surface, it is preferred that a nonskid sole 70 or other application be made upon the sole to provide the necessary traction and friction. In the embodiment shown, the nonskid sole comprises a piece of rubber coated material that is attached to the bottom of the sole through use of an adhesive. However, other nonskid pads or finishes could comprise or be applied to the bottom surface of the sole so as to create “a nonskid sole,” including but not limited to ultra suede, adhesives, rubber, plastics, sprays, “painted on” materials, etc. Optionally, the sole could be made of such material thereby eliminating the need for a separate nonskid sole.

The foot covering 10 further comprises a front portion 30. This front portion extends from a top 32 to a bottom 34. This foot cover's front portion is configured for orientation towards the front or shin side of a wearer's leg. It is preferred that the front portion 30 be separate from the upper front portion 50 because it allows for a better seam to be formed between the two portions. Without such a seam, the foot cover may be inclined to bunch at the bend at the front of the ankle, the bend/bunching forming an additional possible location of wear against the skin of the wearer, an undesirable feature. While the present invention could be differently crafted, cut, and/or molded to accomplish this same purpose, it is envisioned that using a front portion 30 separate from the upper front portion 50 best achieves the goal of providing a foot covering having minimal points of contact with a wearer's sensitive skin.

The figures further show the present invention's rear portion 40. This rear portion 40 has a top 42 and a bottom 44. This rear portion 40 is configured to be oriented on the user at the back or calf side of the user's leg. While the front and rear portions could be formed of the same material, it is preferred that the rear portion extend from above the calf to the heel, thereby providing vertical support for the structure of the foot covering. This front portion 30 and rear portion 40 are configured for attachment together in order to define a “leg portion” of the invented foot covering. In the preferred embodiment, this “attachment together” is accomplished by the front portion 30 having a front portion top 32 extending to a front portion bottom 34, as well as a rear portion 40 extending to a rear portion top 42 and a rear portion bottom 44. These portions are sewn together to form the “leg portion.”

FIGS. 1 and 2 further show the upper front portion 50. The upper front portion serving as the covering of the top of the wearer's foot, cooperating with the leg portion (front and rear) and the sole to form the present invention's preferred embodiment.

It is preferred that the various components of the present invention be formed separately and sewn together using a flat lock seam. However, other manners of construction are likewise envisioned and available, including other sewing methods, adhesives, welding or forming one or more of said components from a single piece of material (for instance, the leg portion could be formed of a tubular portion of the appropriate fabric). What is generally important in the preferred embodiment of the present invention is that the seams formed are external (not within the interior portion of the foot covering). As mentioned before in this disclosure, diabetics and other individuals can have extremely sensitive (to touch) lower extremities. By removing the seams from the inside of the foot covering, the comfort to the wearer is greatly increased because the seam does not irritate the wearers skin.

It is preferred that the present invention be made generally of a felt or fleece style material, such a material providing warmth, as well as being soft to the touch. An example of a suitable fabric is spun fleece or “polar fleece.” Such material is preferred because it insulates the lower extremity, thereby helping retain heat and furthering the present invention's benefits.

It is preferred that the top most portion of the present invention be provided with an adjustable attachment 60. In the embodiment shown in FIGS. 1-3, this adjustable attachment comprising a hook and loop style (Velcro®) fastener that can be used to adjust the relationship between the front portion top 32 and a rear top portion 42. A zipper, string ties, buttons or other manners of closing/constricting the open first end could also be used.

The goal of the adjustable attachment being herein to restrict or constrict the size of the top opening therein to a size that is smaller than the diameter of the wearer's calf, so that essentially the calf of the wearer holds the invented foot and leg warmer up. While it is preferred that the present invention be configured to extend past the user's calf and attach thereto, it is likewise envisioned that similar structure could be provided for holding the sock up around a user's ankle or other body part.

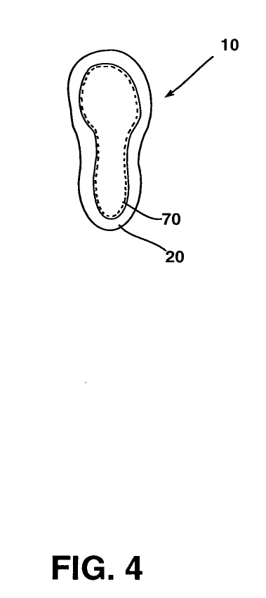

Specifically referring to FIG. 2, shown is a second embodiment of the present invention. This embodiment of the present invention shows a foot and leg warmer 10 having a sole portion 20, which has a nonskid sole 70 (further shown in FIG. 4) applied thereto. While the utilization of such a nonskid sole is preferred, it is not necessary for the operation of the present invention. In FIG. 2, the junction is left unsown and instead being provided with hook and loop style fasteners thereby allowing a user to open and close the side of the foot covering 10. This is especially important where the user has large legs and feet, thereby providing them with an easier way of putting on and taking off said foot and leg warmer 10. Such an embodiment has a side closure 80, preferably comprising one or more strips of hook and loop style fasteners for allowing the side wall to be closed. Optionally, a zipper could be used. The preferred embodiment does not have such a side closure.

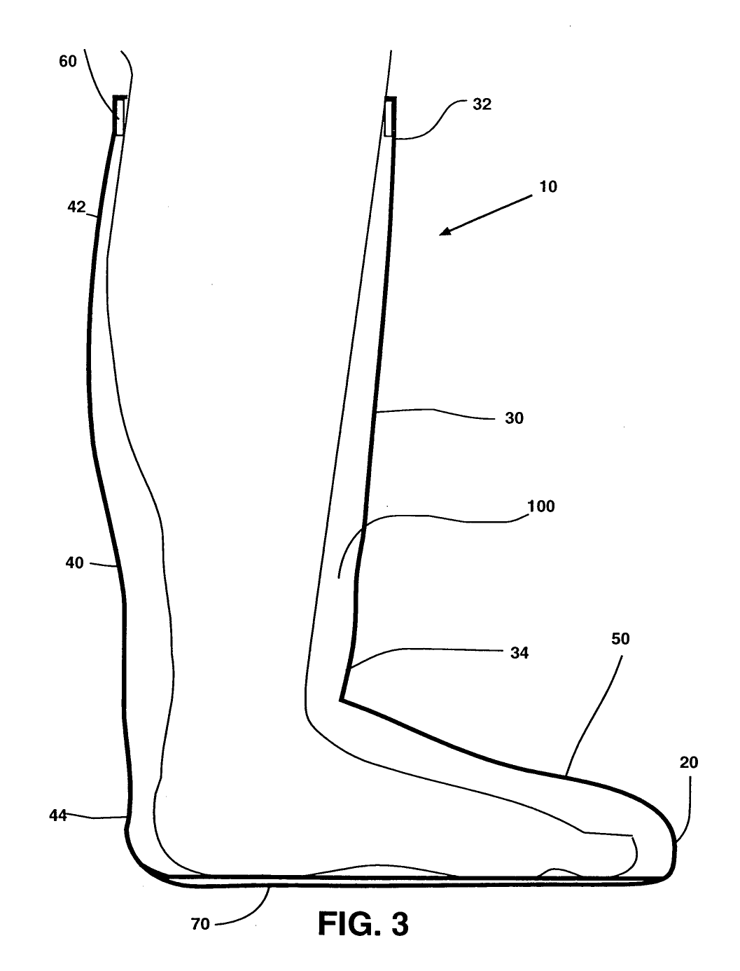

Referring to FIG. 3, it is preferred that the top opening be constricted through use of the adjustable attachment/closure 60 to be barely snug around the wearer's leg, just above the calf. In doing so, a quantity of air 100 is sealed within the foot covering. It is preferred that the foot covering be configured for being loose around the foot/lower extremity of the wearer and thereby a large quantity of air is sealed 100 therein. Being loose itself is not obvious for leg coverings because leg coverings (socks, boots, etc.) are typically configured for fitting snuggly so as to prevent the leg covering from causing a wearer to trip or to prevent shifting or bunching up of the leg covering on the wearer's leg. In the preferred embodiment, the foot covering has an inside surface and an outside surface, said foot covering portions being stitched together on the outside surface so as to not create internal stitched seams within the foot covering.

Individuals with poor circulation in their extremities tend to have reasonable circulation in their calf muscles and occasionally other portions of their lower legs. The present invention can thus be used to increase circulation in the wearer's feet and toes through use of the heat present in the calf muscle (lower leg). Essentially, the calf muscle (and/or lower leg) heats the quantity of air trapped therein, as well as the adjacent fabric and flesh, and this trapped heat and hot air is then used to increase the temperature in the wearer's feet and toes.

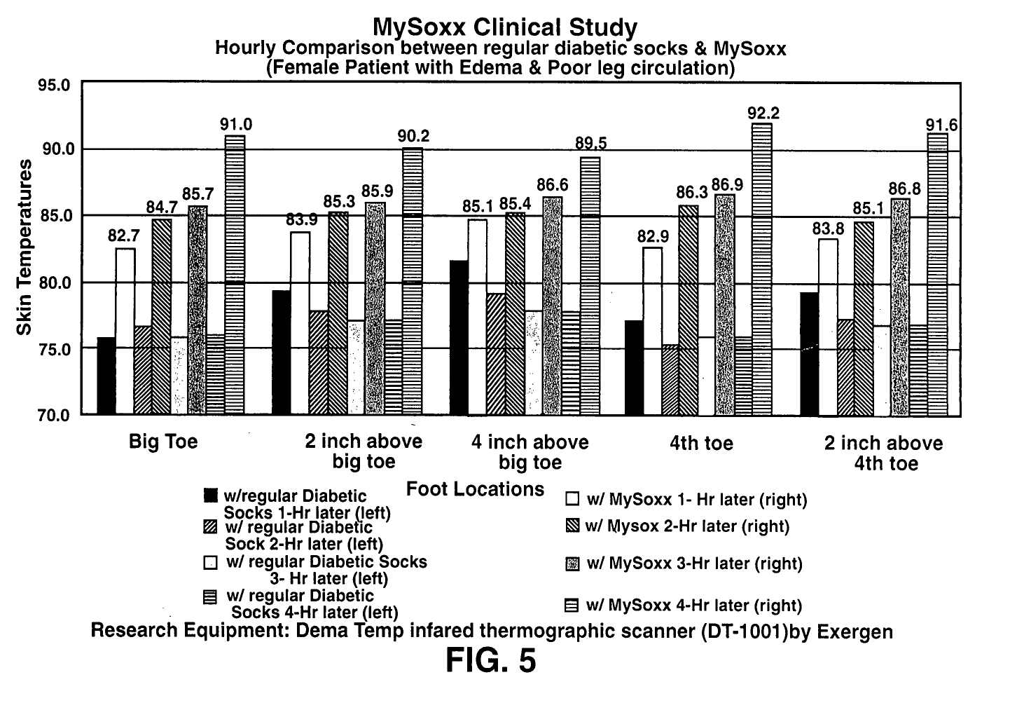

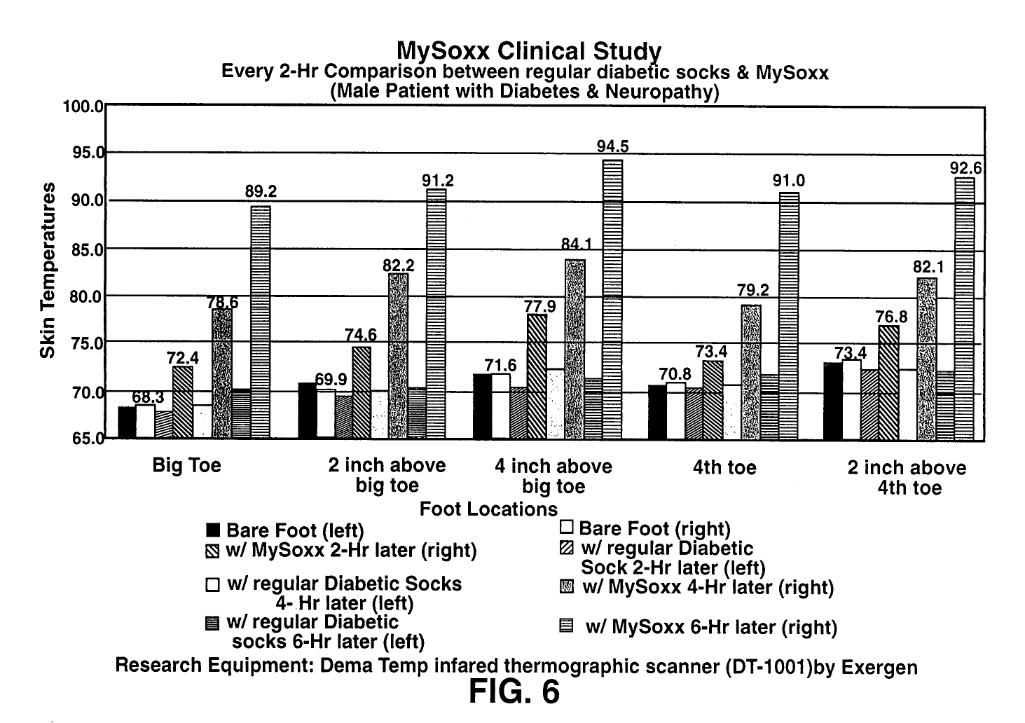

FIGS. 5 and 6 show a pair of clinical studies showing the effect of wearing one of the invented foot coverings on a pair of individuals in comparison to wearing a standard “diabetic sock” and going bare foot. In FIG. 5, a female patient having edema and poor leg circulation is shown raising the temperature of her extremities up to fifteen degrees over wearing just a standard tight fitting “diabetic sock.” In FIG. 6, a male patient having diabetes and neuropathy is shown raising the temperature of his lower extremities upwards of twenty degrees over having bare feet.

Using an adjustable attachment, the user is able to configure the invented foot and leg warmer as tight as the user wants. Some users will not have pain problems with constriction and may choose to have the sock held on tightly, whereas others may desire to have the sock held on very loosely. As such, adjustable tightness in this component as well as in with others may be desirable.

The present invention works best when it is specially contoured to fit (loosely) the shape of the foot of the person who is wearing it. As such, the inventor envisions in one embodiment to utilize a custom four-point design pattern for creating near custom foot covers for his customers. This custom four-point design pattern includes the wearer's shoe size, measurement above the ankle, measurement of the wearer's calf, as well as the heel to upper calf measurement. By taking these measurements and configuring the present invention, a perfect embodiment can be arrived at for each individual wearer.

While there is shown and described the present preferred embodiment of the invention, it is to be distinctly understood that this invention is not limited thereto but may be variously embodied to practice within the scope of the following claims. From the foregoing description, it will be apparent that various changes may be made without departing from the spirit and scope of the invention as defined by the following claims.

1. Method of improving circulation in a diabetic person's foot through using the warmth of the person's calf muscle to warm said foot, said method comprising the steps of:
providing a foot covering for said person's foot, said foot covering configured for loosely fitting said foot thereby defining an air space generally surrounding said foot, said foot covering configured for extending above the person's calf muscle, said foot covering having an open first end extending to a closed second end;
inserting said foot in said foot covering;
constricting said foot covering at or near said first end above said person's calf muscle and thus around said person's lower leg inserted in said foot cover thereby trapping a quantity of air in said airspace; and
using the body heat of the person's calf muscle to warm said trapped air,
wherein said warmed, trapped air fills said air space and results in the gradual warming of the person's foot thereby improving blood circulation in said person's foot.

2. The method of claim 1, wherein said foot covering comprises polar fleece material.

3. The method of claim 2, wherein the foot covering comprises a sole having a non-skid surface applied there-to.

4. The method of claim 1, wherein the step of constricting said foot covering at or near said first end above said person's calf muscle and thus around said person's lower leg inserted in said foot cover thereby trapping a quantity of air in said airspace is accomplished through use of a hook and look style fastener used to close said open first end around said person's leg above said person's calf muscle.

5. The method of claim 1, wherein said foot covering comprising:
a sole portion for generally covering the sole of a wearer;
a front portion for covering a wearer's shin area, said front portion having a top and a bottom;
a rear portion for covering a wearer's calf area, said rear portion having a top and a bottom; and
an upper front portion for covering the top of a wearer's foot;
wherein said sole, said front portion, said rear portion, and said upper front portion are sewn together to form said foot covering.

6. The method of claim 5, wherein said foot covering has an inside surface and an outside surface and wherein said portions are stitched together on the outside surface of said foot covering so as to not create internal stitched seams within the foot covering.

7. Method of improving circulation in a diabetic person's foot through using the warmth of the person's calf muscle to warm said foot, said method comprising the steps of:
providing a polar fleece foot covering for said person's foot, said foot covering configured for loosely fitting said foot thereby defining an air space generally surrounding said foot, said foot covering configured for extending above the person's calf muscle, said foot covering having an open first end extending to a closed second end, said foot covering having a non-skid sole applied thereto;
inserting said foot in said foot covering;
constricting said foot covering at or near said first end above said person's calf muscle and thus around said person's lower leg inserted in said foot cover thereby trapping a quantity of air in said airspace; and
using the body heat of the person's calf muscle to warm said trapped air,
wherein said warmed, trapped air fills said air space and results in the gradual warming of the person's foot thereby improving blood circulation in said person's foot.

8. The method of claim 7, wherein the step of constricting said foot covering at or near said first end above said person's calf muscle and thus around said person's lower leg inserted in said foot cover thereby trapping a quantity of air in said airspace is accomplished through use of a hook and look style fastener used to close said open first end around said person's leg above said person's calf muscle.

9. The method of claim 7, wherein said foot covering comprising:
a sole portion for generally covering the sole of a wearer;
a front portion for covering a wearer's shin area, said front portion having a top and a bottom;
a rear portion for covering a wearer's calf area, said rear portion having a top and a bottom; and
an upper front portion for covering the top of a wearer's foot;
wherein said sole, said front portion, said rear portion, and said upper front portion are sewn together to form said foot covering.

10. The method of claim 9, wherein said foot covering has an inside surface and an outside surface and wherein said portions or stitched together on the outside surface of said foot covering so as to not create internal stitched seams within the foot covering.

11. Method of improving circulation in a diabetic person's foot through using the warmth of the person's calf muscle to warm said foot, said method comprising the steps of:
providing a foot covering for said person's foot, said foot covering configured for loosely fitting said foot thereby defining an air space generally surrounding said foot, said foot covering configured for extending above the person's calf muscle, said foot covering having an open first end extending to a closed second end, said foot covering comprising:
a sole portion for generally covering the sole of a wearer, said sole portion having a non-skid material applied there-to;
a front portion for covering a wearer's shin area, said front portion having a top and a bottom;
a rear portion for covering a wearer's calf area, said rear portion having a top and a bottom; and
an upper front portion for covering the top of a wearer's foot;
wherein said sole, said front portion, said rear portion, and said upper front portion are sewn together to form said foot covering;
inserting said foot in said foot covering;
constricting said foot covering at or near said first end above said person's calf muscle through use of a hook and loop style attachment and thus around said person's lower leg inserted in said foot cover thereby trapping a quantity of air in said airspace; and
using the body heat of the person's calf muscle to warm said trapped air,
wherein said warmed, trapped air fills said air space and results in the gradual warming of the person's foot thereby improving blood circulation in said person's foot;
wherein said foot covering comprises polar fleece material.

12. The method of claim 11, wherein said foot covering has an inside surface and an outside surface and wherein said portions or stitched together on the outside surface of said foot covering so as to not create internal stitched seams within the foot covering.
